(19) United States
(12) Patent Application Publication
(10) Pub. No.: US 2025/0345777 A1
(43) Pub. Date: Nov. 13, 2025

(54) BISMUTHENE AS VERSATILE PHOTOCATALYST OPERATING UNDER VARIABLE CONDITIONS FOR PHOTOREDOX C-H BOND FUNCTIONALIZATION

(71) Applicant: KOC UNIVERSITESI, Istanbul (TR)

(72) Inventors: Onder METIN, Istanbul (TR); Melek Sermin OZER, Istanbul (TR); Zafer EROGLU, Istanbul (TR)

(73) Assignee: KOC UNIVERSITESI, Istanbul (TR)

(21) Appl. No.: 18/862,567

(22) PCT Filed: May 20, 2022
(86) PCT No.: PCT/TR2022/050454

§ 371 Date: Nov. 4, 2024

(51) Int. Cl. B01J 23/18; B01J 35/39; B01J 37/00; B01J 37/16; B01J 37/34; C07C 17/281; C07C 201/12; C07C 253/30; C07D 207/327; C07D 207/33; C07D 207/333; C07D 207/337; C07D 307/36; C07D 307/38; C07D 307/42; C07D 307/54; C07D 333/10; C07D 333/12; C07D 333/16; C07D 333/18; C07D 333/24; C22B 30/06

(52) U.S. Cl. CPC B01J 23/18; B01J 35/39; B01J 37/0018; B01J 37/16; B01J 37/343; C07C 17/281; C07C 201/12; C07C 253/30; C07D 207/327; C07D 207/33; C07D 207/333; C07D 207/337; C07D 307/36; C07D 307/38; C07D 307/42; C07D 307/54; C07D 333/10; C07D 333/12; C07D 333/16; C07D 333/18; C07D 333/24; C22B 30/06

(57) ABSTRACT

A method for the synthesis of two-dimensional (2D) bismuth (bismuthene) and a use of this material as a photoredox catalyst are provided. The 2D bismuthene is prepared by a liquid-phase exfoliation of the 3D-layered bismuth. The photoredox catalyst is used for the C-H functionalization reactions for the synthesis of complex molecules under versatile conditions.

### CROSS REFERENCE TO THE RELATED APPLICATIONS

This application is the national phase entry of International Application No. PCT/TR2022/050454, filed on May 20, 2022, the entire contents of which are incorporated herein by reference.

### TECHNICAL FIELD

The present invention relates to a novel and practical method for the synthesis of two-dimensional (2D) bismuth (bismuthene) and the usage of this material as a photoredox catalyst for the direct C-H functionalization of heteroarenes and arenes under versatile conditions including indoor light illumination, darkness, outdoors and low temperature.

### BACKGROUND

As the monolayer of 2D pnictogens exhibits increased band gap with tunable electronic and optical properties, they are promising materials to be used as photocatalysts in different reactions. Among them, bismuthene, mono/few layer(s) of three-dimensional (3D) layered bismuth, possessing excellent electron mobility properties, low toxicity, and high stability, exhibits unique potential for photocatalytic applications owing to its 2D hexagonal lattice, narrow optical band gap falling into the far visible/NIR wavelength, and high surface area. In addition to these favorable assets, bismuthene stands as a suitable photocatalytic material because it shows semiconducting properties with a tunable band gap of 0.3-1.0 eV when the exfoliated layers are thinner than 30 nm. To the best of our knowledge, there is no example of using bismuthene as a photocatalyst in any liquid-phase chemical transformation so far.

When the photocatalysts reported for C-H functionalization in the literature is surveyed, it will be seen that they have mostly homogeneous nature including transition metal complexes and small organic dye molecules in the reaction medium. Although some have photocatalytic nature offering environmentally friendly methodologies, metal traces may remain in drugs, as they are difficult to remove from desired products. Additionally, photocatalytic ones require extra light sources with complex reaction setups, which complicates the reproducibility of a given procedure. In addition, other necessary parts of a molecule can be reduced/oxidized by the photocatalyst present under the given light source or the elevated reaction temperature.

In the prior art, there is no solution demonstrating a photocatalyst that can be operated under versatile conditions and having a heterogeneous photocatalytic nature along with being reusable as the bismuthene possesses.

### SUMMARY

The objective of the present invention is to provide a new method (wet-chemical/protocol) for the synthesis few-layer bismuthene with adjustable thickness and tunable bandgap. The development of a wet-chemical method for the preparation of relatively small sized 3D-layered bismuth compared to the top-down methods is important for obtaining high yield bismuthene nanosheets via liquid-phase exfoliation process. The studies carried out in the scope of the invention, to understand the underlying reason behind the high efficiency of the C-H arylation reaction by bismuthene under versatile conditions, it was investigated experimentally and single electron transfer from bismuthene to diazonium salts was found to be a radical mechanism. These mechanistical experiments are consistent with the property of bismuthene (narrow bandgap, 0.6 eV), in which can catalyze the reaction without an external light source. After proving photocatalytic nature of bismuthene on a photocatalytic application for the first time, the applicability of this catalyst was further expanded on the C-H arylation with different substrates. Bismuthene nanosheets were applied on a large substrate scope (43 examples in total) for the application of bismuthene-based photoredox catalysis over a variety of heteroarenes (furan, thiophene and pyrrole) and, more importantly arenes (benzene and nitrobenzene) with aryl diazonium salts.

Bismuthene is a complementary photocatalyst for C-H functionalization as it tolerates redox active functional groups of a reactant, because even at lower temperatures it can transfer single electron to diazonium salts. The operation of bismuthene under versatile conditions can easily tolerate various important parts of a sensitive molecule, enabling successful synthesis of late- stage complex structures.

The objective of the present invention is to provide low cost and reusable photocatalyst that does not require a dry solvent, inert atmosphere or even a light source for photoredox C-H functionalization. The state-of-the-art photoredox catalysts for the photoredox C-H functionalization is mainly based on either transition metal complexes or organic dye molecules which are soluble in the reaction medium and thus they have a recovery problem and require external light sources and long reaction times to be activated. Unlike other existing methodologies, bismuthene can be operated, with high product yields, in a variety of reaction conditions including indoor light irradiation, dark, outdoors, and low temperature, which is unique property for a photocatalyst.

The objective of the present invention is to report for the first time that bismuthene is a versatile photoredox catalyst for the direct C-H arylation of (hetero)arenes under versatile conditions including indoor light illumination, darkness, outdoors and low temperature.

### BRIEF DESCRIPTION OF THE DRAWINGS

“BISMUTHENE AS A VERSATILE PHOTOCATALYST OPERATING UNDER VARIABLE CONDITIONS FOR THE PHOTOREDOX C-H BOND FUNCTIONALIZATION” developed to fulfill the objectives of the present invention is illustrated in the accompanying figures, in which;

FIGS. 1A-1G: Morphological characterization of bismuthene. FIG. 1A is SEM image of bismuthene, FIG. 1B is TEM image of bismuthene, FIG. 1C is AFM image of bismuthene deposited on high quality silicon wafer, FIG. 1D shows the corresponding height profiles for three bismuthene nanosheets marked in FIG. 1C, FIG. 1E is Typical HAADF-STEM image of bismuthene, FIG. 1F shows Bi elemental mapping, FIG. 1G shows O elemental mapping. (Scale bars in are all 200 nm.)

FIGS. 2A-2F: Chemical, physical, optical, and electrochemical characterization of bismuthene. FIG. 2A is XRD pattern of 3D-layered Bi, FIG. 2B is High-resolution XPS spectrum of Bi 4f core-level of bismuthene, FIG. 2C is Raman spectrum of bulk Bi and bismuthene deposited on ITO, FIG. 2D is Vis-NIR absorption spectrum of bismuthene in DMSO, FIG. 2E is Mott-Schottky plot of bismuthene, FIG. 2F is XPS valance band spectra.

FIGS. 3A-3B: Stability experiments. FIG. 3A shows XRD patterns and FIG. 3B shows XPS of 2D bismuthene after 60 days.

FIGS. 4A-4B: Surface composition experiment. FIG. 4A is XPS Survey spectrum of bismuthene and FIG. 4B is High-resolution XPS spectrum of Bi 5d core-level of bismuthene.

FIG. 5: The energy band alignment of bismuthene.

FIGS. 6A-6B: Results of the experiments. FIG. 6A shows scavenger experiments, FIG. 6B shows plausible reaction mechanism.

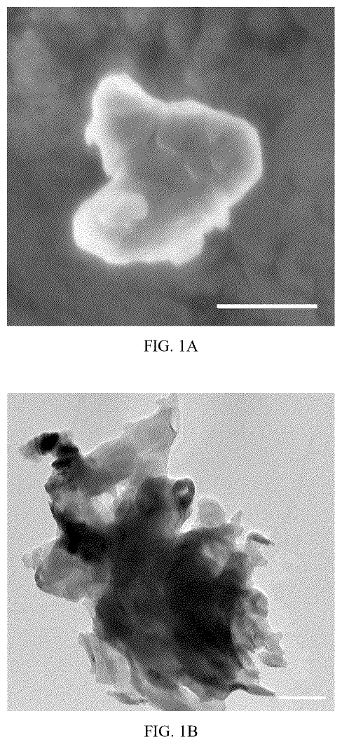

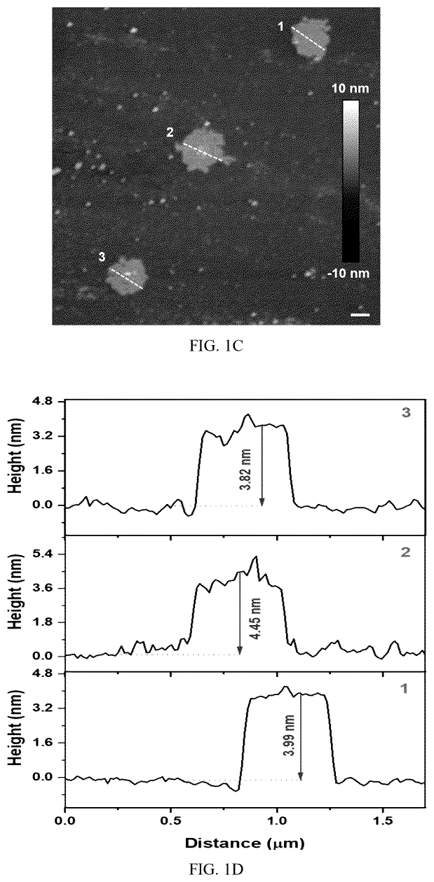

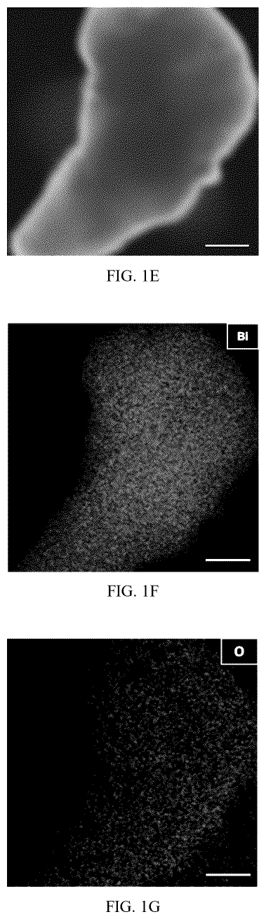

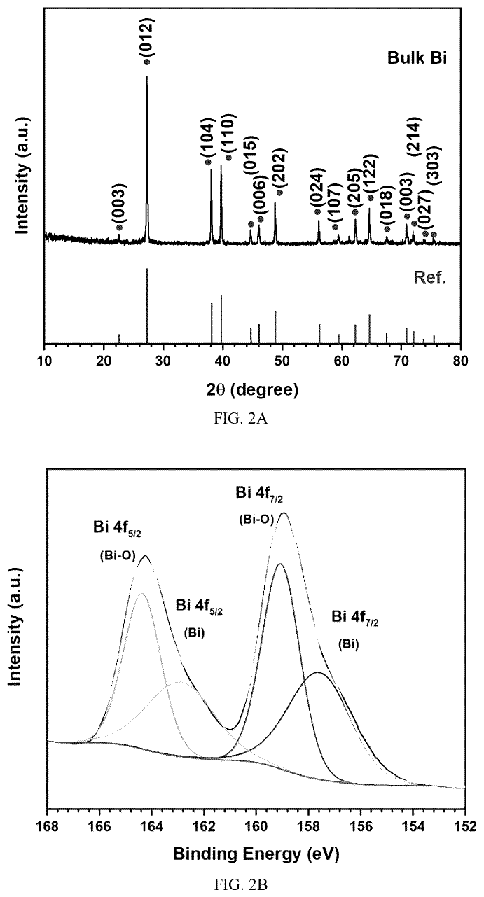

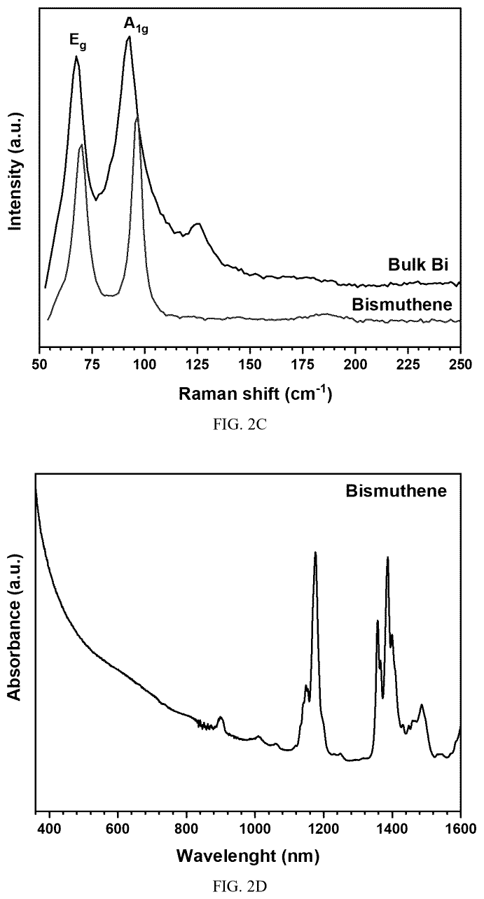

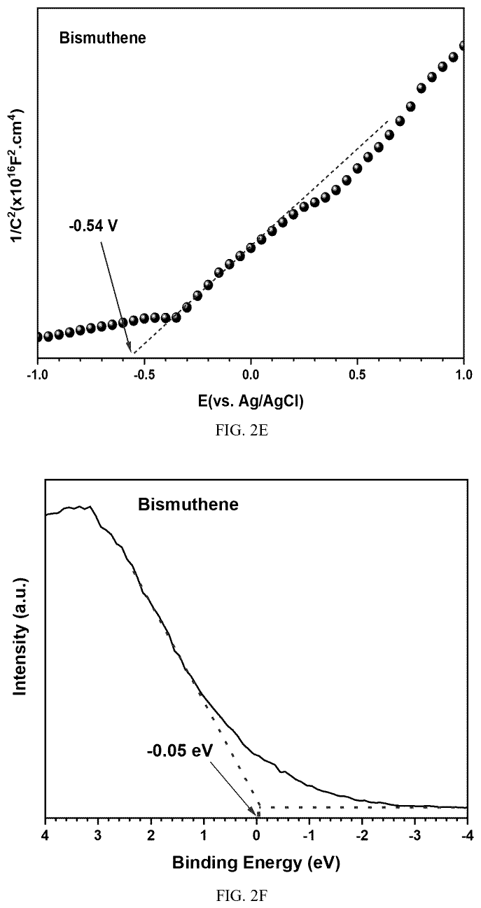

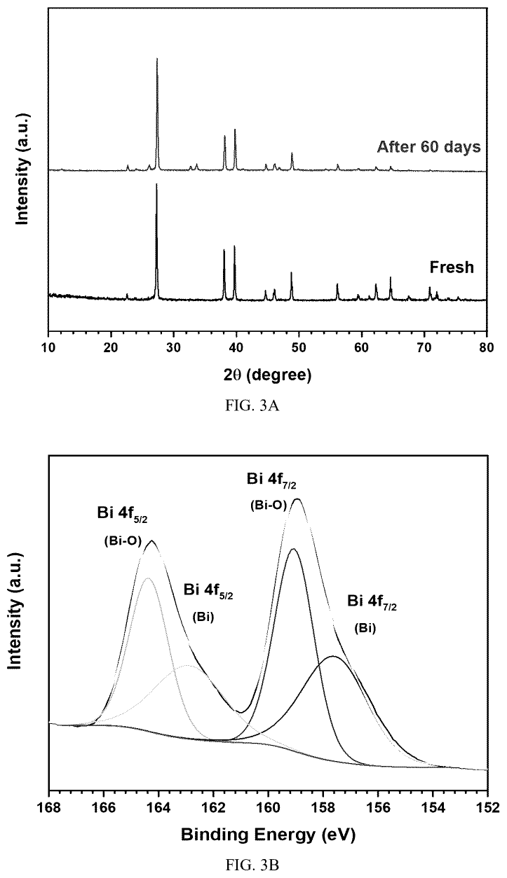

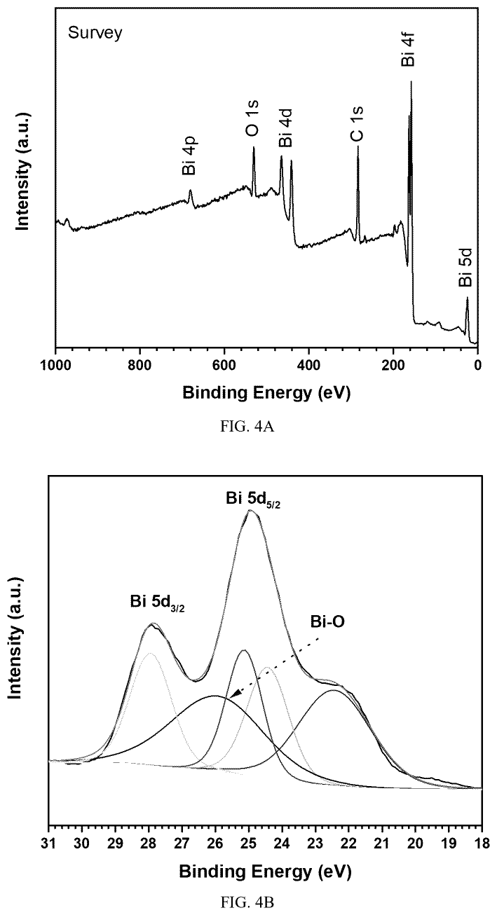

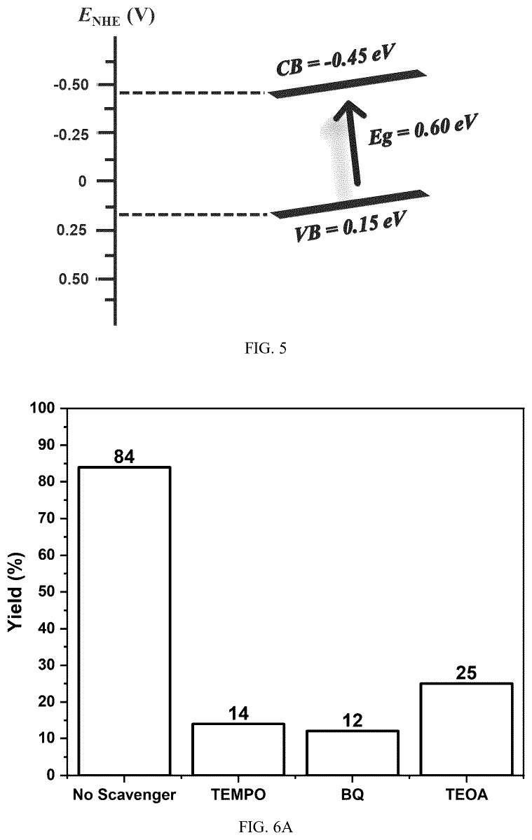

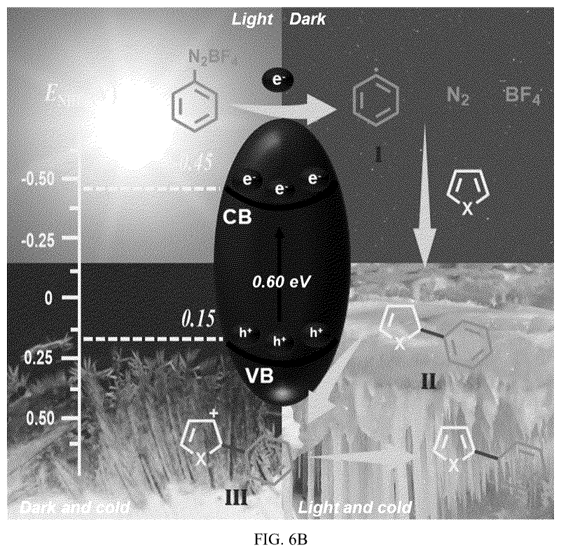

### DETAILED DESCRIPTION OF THE EMBODIMENTS

The present invention relates to a new method for the synthesis of bismuthene and usage of this material as a photoredox catalyst for the C-H functionalization reactions for the synthesis of complex structure under ambient conditions. The first step of the method is the synthesis of 3D-layered Bi. In the first step, bismuth (III) chloride (BiCl₃) is reduced into 3D-layered Bi via surfactant-assisted chemical reduction (SACR) method. SACR method is a general procedure for the synthesis of materials which includes surfactant type, surfactant concentration, reducing agent, synthesis method, solvent, and temperature. By this methodology, the parameters such as the shape, size, thickness, and layers can be controlled even in large-scale production. In the invention, 3D-layered Bi was synthesized for the first time via SACR method by using oleylamine (OAm) as both surfactant and solvent, borane tert-butylamine (TBAB) as a reducing agent under mild conditions. Detailed procedure for the synthesis of 3D-layered Bi is as follows.
BiCl₃ (1.0 mmol) was added into a 100 mL of four-necked round bottom flask with magnetic stirring bar under argon gas flow.
OAm (10.0 mL) was poured and stirred while it is attached to a continuous electronic temperature control via a thermocouple immersed in the solution and heated up to 120° C.
The current Argon flow was stopped, right after replaced with vacuum controller and kept at this temperature for 30 min.
TBAB (1.5 mmol) which dissolved in OAm (1.5 mL) via sonication was injected into the resulting BiCl₃-OAm solution.
The mixed solution was stirred for 30 min at 120° C. and cooled naturally to room temperature.
The resultant black precipitate was separated from the solution by centrifugation and washed with ethanol three times.
The obtained powders were dried under vacuum at 40° C. and then stored.

By the exfoliation process, 2D bismuthene nanosheets are obtained from the 3D layered bismuth as the followings.
10 mg of Bulk bismuth was exfoliated in 1 ml of solvent such as dimethyl sulfoxide (DMSO), dimethyl formamide (DMF), ethyl acetate (EtOAc), N-methyl-2-pyrrolidone (NMP), deionized water (DI H₂O), Acetone, 2-propanol (i-PrOH), and acetonitrile (MeCN) under ambient conditions by ultrasonic homogenizer using a Bandelin Sonoplus 2200.2, 200 W, 25% amplitude,
The dispersed bismuthene nanosheets were further used for photoredox C-H arylation.

The representative steps for the synthesis of bismuthene is given below:

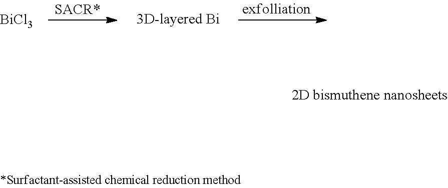

After the exfoliation of 3D-layered Bi, the obtained nanosheets can be used as photoredox catalyst in the C-H arylation of furan with 4-chlorobenzenediazonium tetrafluoroborate as a model reaction. General procedure for photoredox C-H arylation as follows.
After exfoliation of the 3D-layered Bi in DMSO (1 mL), the corresponding suspension and 1 mL of heteroarene (furan, thiophene, or N-Boc pyrrole) or 1 mL of arene (benzene or nitrobenzene) were added to the 25 mL of jacketed flask with a stirring bar and a septum.
Subsequently, the corresponding aryl diazonium salt (0.25 mmol) was added to the solution one pot as solid. The jacketed flask was kept at the required conditions (in light/dark or with white light source (150 W) etc.).
Condition A: After 2 h of stirring the reaction mixture was transferred to separating funnel, diluted with dichloromethane (DCM) and washed with water (3×10 mL). Then the organic layer was separated, dried over Na₂SO₄, filtered and concentrated.
Condition B: After 5 h of stirring the reaction mixture was transferred to separating funnel, diluted with DCM and washed with water (3×10 mL). Then the organic layer was separated, dried over Na₂SO₄, filtered and concentrated.
The residue was purified by column chromatography on SiO₂ using EtOAc/n-hexane as eluent if needed.

One example of this functionalization is given below:

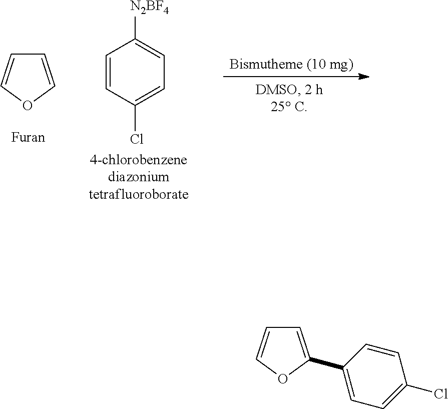

This method and the unique properties of the photocatalyst (sustainability, reusability, and operability under versatile conditions) allow the appropriate synthesis of late-stage sensitive structures highly possible. In other words, no light requirement or temperature-independent nature of bismuthene could be a unique example for the development of a non-stop C-H arylation process in industry under daylight or dark. The catalyst obtained in this invention will be a great candidate for the production of other organic molecules with similar structures. These results will give opportunity to develop the applications (water splitting, hydrogen evolution, degradation of organic pollutants etc.) of bismuthene even further as this semiconductor can be activated by near infrared irradiation.

The reaction yield in the presence of bismuthene at the specified conditions is given in the table below.

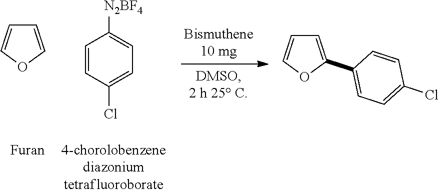

| Entry | Conditions^{a} | Yield [%]^{b} |
|---|---|---|
| 1 | Furan (10 equiv), DMSO, 25° C. | 63 |
| 2 | Furan (20 equiv), DMSO, 25° C. | 75, 67^{c}, 77^{d} |
| 3 | Furan (50 equiv), DMSO, 25° C. | 84, 80^{e} |
| 4 | Furan (20 equiv), DMF, MeCN, | 45, 38, 14, 25, |
| | NMP, H₂O, Acetone, 25° C. | Trace |
| 5 | Furan (20 equiv), DMSO, 25° C., 5 mg | 69 |
| | catalyst | |
| 6 | Furan (20 equiv), DMSO, 25° C., | Trace |
| | no catalyst | |
| 7 | Furan (20 equiv), DMSO, 25° C., bulk Bi | 36 |
| 8 | Furan (20 equiv), DMSO, 25° C., DARK | 76 |
| 9 | Furan (20 equiv), DMSO, 5° C. | 76 |
| 10 | Furan (20 equiv), DMSO, 5° C., DARK | 75 |

^{a}(All reactions were carried out with 10 mg catalyst on a scale of 5 mmol of Furan
and 0.25 mmol of 4-chlorobenzenediazonium tetrafloroborate in 1 mL of solvent
at 25° C. Indoor light for 2 h unless otherwise noted.);
^{b}(Yields were determined by ¹H NMR analysis with 1,3-dinitrobenzene as internal
standard);
^{c}(Reaction time is 1 h.);
^{d}(Reaction time is 4 h.);
^{e}(Isolated yield.) (DARK: The reaction setup was covered with aluminum foil,
absence of indoor/outdoor light.)

Scope and limitation of diazonium salts in the bismuthene catalyzed photoredox C-H arylation of various heteroarenes and arenes are given in the table below.

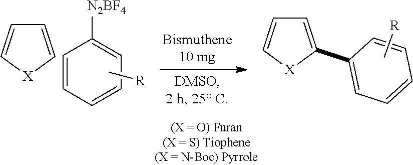

a. C—H arylation of Furan with condition A

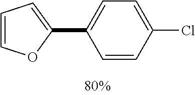

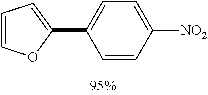

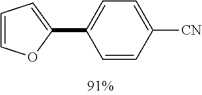

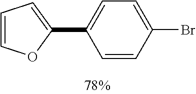

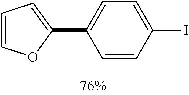

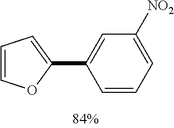

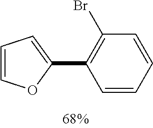

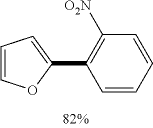

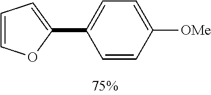

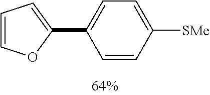

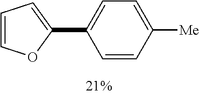

b. C—H arylation of Thiophene with condition B

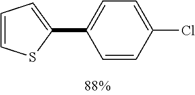

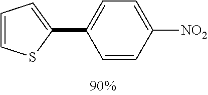

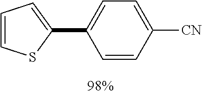

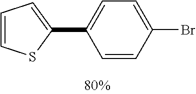

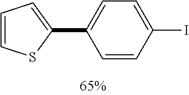

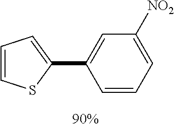

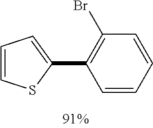

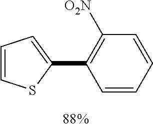

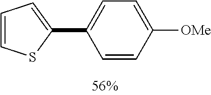

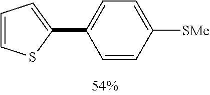

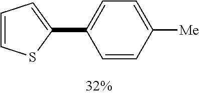

c. C—H arylation of N-Boc Pyyroles with condition A

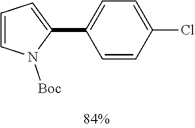

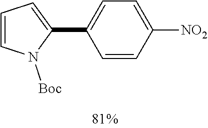

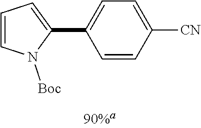

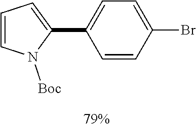

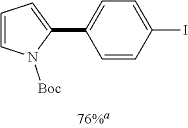

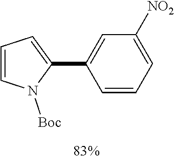

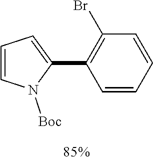

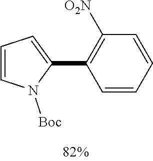

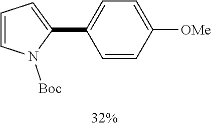

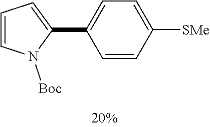

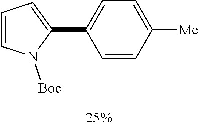

d. C—H arylation of arenes and other heteroarenes with condition B

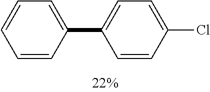

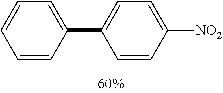

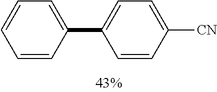

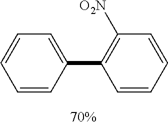

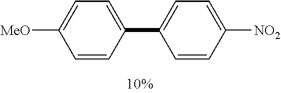

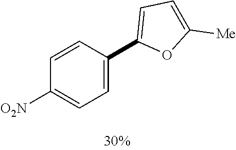

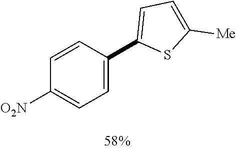

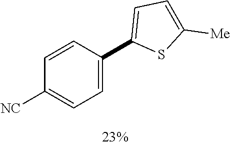

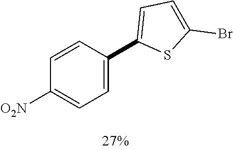

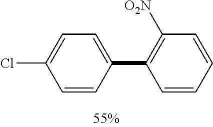

Temperature/light experiments and the effect of the light is given table below:

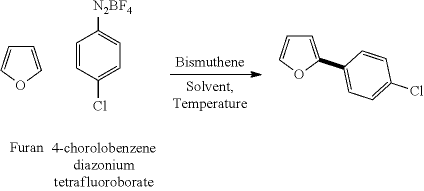

| Entry | Conditions^{a} | Yield [%]^{b} |
|---|---|---|
| 1 | DMF, 25° C. | Trace^{c}, 44 |
| 2 | DMF, 25° C., DARK^{d} | 48 |
| 3 | DMF, 25° C., white light | 43, Trace^{c} |
| 4 | DMF, 5° C. | Trace |
| 5 | DMF, 5° C., DARK^{d} | Trace |
| 6 | DMF, 5° C., white light | 34 |
| 7 | DMF, −15° C. | Trace |
| 8 | DMF, −15° C., DARK^{d} | No rxn |
| 9 | DMF, −15° C., white light | 72 |

^{a}(All reactions were carried out with 10 mg catalyst on a scale of 5 mmol of Furan
and 0.25 mmol of 4-chlorobenzenediazonium tetrafloroborate in 1 mL of solvent
at 25° C. indoor light for 2 h unless otherwise noted.);
^{b}(Yields were determined by ¹H NMR analysis with 1,3-dinitrobenzene as internal
standard);
^{c}(In the absence of the catalyst.);
^{d}(DARK: The reaction setup was covered with aluminum foil, absence of indoor/
outdoor light.).

What is claimed is:

1. A method for synthesizing bismuthene, comprising steps of:
applying a surfactant-assisted chemical reduction method comprising oleylamine as a surfactant and a solvent, borane tert-butylamine as a reducing agent to obtain 3D-layered bismuth from bismuth (III) chloride (BiCl₃),
preparing 2D bismuthene from the 3D-layered bismuth by using a liquid-phase exfoliation method.

2. The method for synthesizing the bismuthene according to claim 1, -wherein the step of preparing the 2D bismuthene is conducted by a liquid-phase exfoliation of the 3D-layered bismuth in dimethyl sulfoxide (DMSO), dimethyl formamide (DMF), ethyl acetate (EtOAc), N-methyl-2-pyrrolidone (NMP), H₂O, acetone, 2-propanol (i-PrOH), and acetonitrile (MeCN) under ambient conditions using a Bandelin Sonoplus 2200.2 ultrasonic homogenizer at 200 W, 25% amplitude for 1 h.

3. A method of using the bismuthene synthesized by the method for synthesizing the bismuthene according to claim 1 as a photoredox catalyst.

4. The method of using the bismuthene according to claim 3, wherein the photoredox catalyst is used in a C-H functionalization of heteroarenes comprising furan, thiophene, and pyrrole in a presence of diazonium salts under dark, outdoor, and indoor lighting at room temperature.

5. The method of using the bismuthene according to claim 3, wherein the photoredox catalyst is used in a C-H functionalization of challenging arenes comprising benzene and nitrobenzene in a presence of diazonium salts under dark, outdoor, and indoor lighting at room temperature.

6. The method of using the bismuthene according to claim 3, wherein in the method for synthesizing the bismuthene, the step of preparing the 2D bismuthene is conducted by a liquid-phase exfoliation of the 3D-layered bismuth in dimethyl sulfoxide (DMSO), dimethyl formamide (DMF), ethyl acetate (EtOAc), N-methyl-2-pyrrolidone (NMP), H2O, acetone, 2-propanol (i-PrOH), and acetonitrile (MeCN) under ambient conditions using a Bandelin Sonoplus 2200.2 ultrasonic homogenizer at 200 W, 25% amplitude for 1 h.
